(19) United States
(12) Patent Application Publication
(10) Pub. No.: US 2019/0375106 A1
(43) Pub. Date: Dec. 12, 2019

(54) ROBOT AND CONTROL METHOD THEREOF

(71) Applicant: LG ELECTRONICS INC., Seoul (KR)

(72) Inventor: Won Hee LEE, Seoul (KR)

(73) Assignee: LG ELECTRONICS INC., Seoul (KR)

(21) Appl. No.: 16/551,589

(22) Filed: Aug. 26, 2019

(30) Foreign Application Priority Data
Jul. 25, 2019 (KR) 10-2019-0090229

(51) Int. Cl. B25J 9/16; A61H 3/00; B25J 3/00; A61F 2/70; B25J 9/00

(52) U.S. Cl. CPC B25J 9/1682; A61H 3/00; B25J 3/00; A61F 2/70; B25J 9/0006; A61H 2201/5097; A61H 2201/5064; A61H 2201/5094; A61F 2002/704; A61H 2003/007; A61H 2201/5007

(57) ABSTRACT

A robot and a method for controlling the robot are provided. The robot may be a wearable robot that may be worn by or removed from the body of the user, and may support the movement of the body of the user. The robot may include a controller, a sensing unit configured to sense a position and pose of a user and transmit the sensed position and pose to the controller, and an actuator configured to receive a command from the controller, operate, and move positions of joints of the robot. The controller may generate motion trajectories of the joints of the robot in order to move the joints of the robot to be arranged at positions of the corresponding joints of the user, based on information on the position and pose of the user received from the sensing unit. The robot system may transmit and receive a wireless signal over a mobile communication network based on 5G communication technologies.

### CROSS-REFERENCE TO RELATED APPLICATION

Pursuant to 35 U.S.C. § 119(a), this application claims the benefit of earlier filing date and right of priority to Korean Patent Application No. 10-2019-0090229, filed on Jul. 25, 2019, the contents of which are hereby incorporated by reference herein in its entirety.

### BACKGROUND

### 1. Technical Field

The present disclosure relates to a robot and a method for controlling the robot, and more particularly, to a robot worn by a user to support movement of the body of the user, and a method of controlling the robot.

### 2. Description of Related Art

The description in this section merely provides background information on embodiments of the present disclosure, and does not constitute prior art.

The use and development of robots in various fields in order to help people have becoming widespread. For convenient use, wearable robots that may be easily worn on and removed from a person's body are being developed.

In particular, wearable robots are being used in order to help movement of a user's body, such as walking and holding heavy objects by the arms. A robot that supports the movement of the body of the user in this manner should be designed such that it may be easily worn and removed by the user.

In particular, a disabled person who has difficulties in using the arms and legs may have considerable difficulty in wearing or removing a robot that helps the movement of the body, and he or she may need the help of another person in order to wear or remove the robot.

Therefore, there is a need for a wearable robot that may be easily worn or removed by the user, without the help of another person, so that a physically disabled person may conveniently use the wearable robot.

In U.S. Patent Application Publication No. US 2014/0100493 A1, a bipedal exoskeleton configured to be worn by a user is disclosed.

In Korean Patent Application Publication No. 10-2015-0053854, a walk-assistive robot and a method for controlling the same are disclosed.

However, in the above documents, a wearable robot that may be worn by or removed from a user without the help of another person is not disclosed.

### SUMMARY OF THE INVENTION

One aspect of the present disclosure is to provide a robot capable of supporting movement of the body of a user and of being easily worn by or removed from the user, without the help of another person.

Another aspect of the present disclosure is to provide a robot capable of guaranteeing safety of a user when the robot is worn by or removed from the user.

The present disclosure is not limited to the aspects described above, and other aspects not mentioned may be more clearly understood from the following description by a person skilled in the art to which the present disclosure pertains.

A robot according to an embodiment of the present disclosure may obtain information on a pose and position of a user by using a plurality of sensing devices, and based on the obtained information may move toward the user and so as to be safely worn by the user.

The robot may obtains information on a pose and position of a user by using a plurality of sensing devices, and based on the obtained information may be safely removed from the user.

The robot may be a wearable robot that may be worn by or removed from the body of the user, and may support the movement of the body of the user.

The robot may include a controller, a sensing unit configured to sense a position and pose of a user and transmit the sensed position and pose to the controller, and an actuator configured to receive a command from the controller, operate, and move positions of joints of the robot.

The controller may be configured to generate motion trajectories of the joints of the robot in order to move the joints of the robot to be arranged at positions of corresponding joints of the user, based on information on the position and pose of the user received from the sensing unit.

The sensing unit may include an RGB camera configured to acquire the position of the user, and a depth camera configured to acquire distances from the robot to the joints of the user.

The robot may further include proximity sensors arranged in the robot and connected to the controller so as to sense whether the respective parts of the robot are within a set distance from the corresponding parts of the body of the user.

The robot may move to the position of the user by walking by itself.

The robot may move to the position of the user by being mounted on a moving device.

The robot may further include a position sensor configured to sense the position the robot. The controller may compensate a distance and direction to the position of the user in first coordinates representing the position of the robot, may calculate second coordinates representing the position of the user, and may generate motion trajectories between the first coordinates and the second coordinates.

A method for controlling a robot according to another embodiment of the present disclosure may include the robot checking a call of a user, the robot moving toward the user, checking whether the robot has reached a target point, and the user wearing the robot.

The robot moving toward the user may include the sensing unit photographing the user, the controller generating motion trajectories of joints of the robot based on an image photographed by the sensing unit, and the robot moving to a target point along the generated motion trajectories of the joints of the robot.

The robot may be removed from the user by a request of the user. The removing of the robot may include the robot checking the removal request of the user, the robot checking whether there is an available space in which the robot may be removed, the robot checking whether the user is safe, the robot being separated from the user, and the robot completely reaching a storage space.

According to embodiments of the present disclosure, since the robot may approach the user and may be worn by the user in accordance with the command of the user, convenience of the user may be improved.

According to the embodiments of the present disclosure, the user may wear the robot without the help of another person.

According to the embodiments of the present disclosure, the user may remove the robot without the help of another person.

According to the embodiments of the present disclosure, the robot may be safely worn by or removed from the user by using the sensing unit.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 is a view illustrating a connection among a wearable robot, a server, and a terminal according to an embodiment of the present disclosure;

FIG. 2 is a view illustrating a structure of a wearable robot according to an embodiment of the present disclosure;

FIG. 3 is a view illustrating a function of a sensing unit according to an embodiment of the present disclosure;

FIG. 4 is a view illustrating an operation of a wearable robot according to an embodiment of the present disclosure;

FIG. 5 is a view illustrating a method of generating motion trajectories of joints of a wearable robot according to an embodiment of the present disclosure;

FIG. 6 is a view illustrating a method of controlling a wearable robot according to an embodiment of the present disclosure;

FIG. 7 is a view illustrating a method of a robot moving toward a user according to an embodiment of the present disclosure;

FIG. 8 is a view illustrating a method of a user wearing a robot according to an embodiment of the present disclosure;

FIG. 9 is a view illustrating a method of a robot being removed from a user according to an embodiment of the present disclosure;

FIG. 10 is a view illustrating a method of a robot checking safety of a user according to an embodiment of the present disclosure;

FIG. 11 is a view illustrating a method of a robot being separated from a user according to an embodiment of the present disclosure;

FIG. 12 is a diagram illustrating an artificial intelligence (AI) device according to an embodiment of the present disclosure;

FIG. 13 is a diagram illustrating an AI server according to an embodiment of the present disclosure; and

FIG. 14 is a diagram illustrating an AI system according to an embodiment of the present disclosure.

### DETAILED DESCRIPTION

Advantages and features of the present disclosure and methods for achieving them will become apparent from the descriptions of aspects herein below with reference to the accompanying drawings. However, the present disclosure is not limited to the aspects disclosed herein but may be implemented in various different forms. The aspects are provided to make the description of the present disclosure thorough and to fully convey the scope of the present disclosure to those skilled in the art. It is to be noted that the scope of the present disclosure is defined only by the claims.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

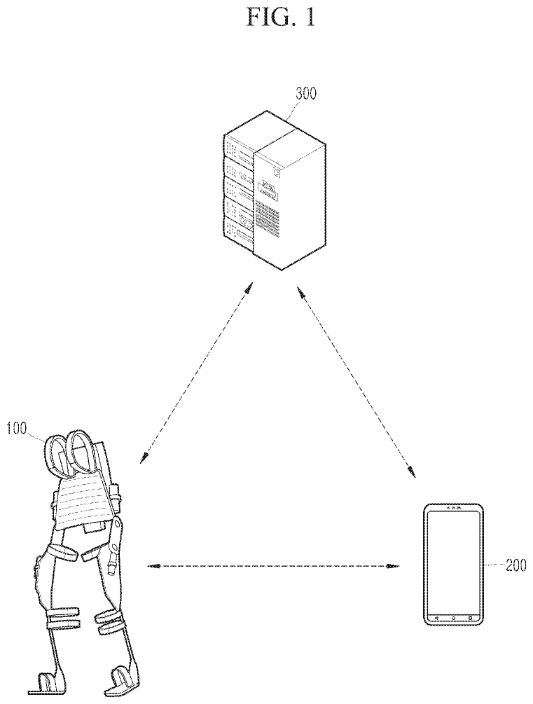

FIG. 1 is a view illustrating a connection among a wearable robot 100, a server 300, and a terminal 200 according to an embodiment of the present disclosure.

A robot may refer to a machine which automatically handles a given task by its own ability, or which operates autonomously. In particular, a robot having a function of recognizing an environment and performing an operation according to its own judgment may be referred to as an intelligent robot.

Robots may be classified into industrial, medical, household, and military robots, according to the purpose or field of use.

A robot may include a driving unit including an actuator or a motor in order to perform various physical operations, such as moving joints of the robot. Moreover, a movable robot may include, for example, a wheel, a brake, and a propeller in the driving unit thereof, and through the driving unit may thus be capable of traveling on the ground or flying in the air.

The wearable robot 100 according to this embodiment is removable from the body of a user, and may support movement of the body of the user.

For example, the wearable robot 100 may be worn by a user with reduced mobility, such as a disabled person or an elderly or infirm person, and the respective parts of the wearable robot 100 may be moved by using an actuator 130 to support the movement of a body part with reduced mobility, such as the arm or the leg of the user.

In order to perform the functions described above, the robot 100 may be provided to be capable of self-driving.

Self-driving refers to a technology in which driving is performed autonomously, and a self-driving vehicle refers to a vehicle capable of driving without manipulation of a user or with minimal manipulation of a user.

For example, self-driving may include a technology in which a driving lane is maintained, a technology such as adaptive cruise control in which a speed is automatically adjusted, a technology in which a vehicle automatically drives along a defined route, and a technology in which a route is automatically set when a destination is set.

A vehicle includes a vehicle having only an internal combustion engine, a hybrid vehicle having both an internal combustion engine and an electric motor, and an electric vehicle having only an electric motor, and may include not only an automobile but also a train and a motorcycle.

In this case, a self-driving vehicle may be considered as a robot with a self-driving function.

Embodiments of the present disclosure may relate to extended reality (XR). XR collectively refers to virtual reality (VR), augmented reality (AR), and mixed reality (MR). VR technology provides objects or backgrounds of the real world only in the form of CG images, AR technology provides virtual CG images overlaid on the physical object images, and MR technology employs computer graphics technology to mix and merge virtual objects with the real world.

MR technology is similar to AR technology in that both technologies involve physical objects being displayed together with virtual objects. However, while virtual objects supplement physical objects in AR, virtual and physical objects co-exist as equivalents in MR.

XR technology may be applied to a head-mounted display (HMD), a head-up display (HUD), a mobile phone, a tablet PC, a laptop computer, a desktop computer, a TV, digital signage, and the like. A device employing XR technology may be referred to as an XR device.

The robot 100 may be communicably connected to the server 300 and the terminal 200. For example, the robot 100 may be connected to the server 300 and may receive software required for an operation from the server 300, and such software may be frequently updated.

The robot 100 may be connected to the terminal 200 and may receive, for example, a command of the user on the wearing or removing of the robot 100 from the terminal 200. Here, the terminal 200 may be a smart phone or a table PC that may be easily carried by the user.

The terminal 200 may be connected to the server 300 and may receive, from the server 300, software required for an operation and control of the robot 100, and such software may be frequently updated.

Artificial intelligence technology may be applied to the robot 100. Artificial intelligence refers to a field of studying artificial intelligence or a methodology for creating the same. Moreover, machine learning refers to a field of defining various problems in the field of artificial intelligence, and studying methodologies for solving the problems. Machine learning may also be defined as an algorithm for improving performance with respect to a task through repeated experience with respect to the task.

An artificial neural network (ANN) is a model used in machine learning, and may refer in general to a model with problem-solving abilities, composed of artificial neurons (nodes) forming a network by a connection of synapses. The ANN may be defined by a connection pattern between neurons on different layers, a learning process for updating model parameters, and an activation function for generating an output value.

The ANN may include an input layer and an output layer, and may selectively include one or more hidden layers. Each layer includes one or more neurons, and the artificial neural network may include synapses that connect the neurons to one another. In an ANN, each neuron may output a function value of an activation function with respect to the input signals inputted through a synapse, a weight, and a bias.

Model parameters refer to parameters determined through learning, and may include weights of synapse connections, biases of neurons, and the like. Moreover, hyperparameters refer to parameters which are set before learning in a machine learning algorithm, and include a learning rate, a number of iterations, a mini-batch size, an initialization function, and the like.

The objective of training an ANN is to determine model parameters for significantly reducing a loss function. The loss function may be used as an indicator for determining an optimal model parameter during the learning process of an artificial neural network.

Machine learning may be classified into supervised learning, unsupervised learning, and reinforcement learning, depending on the learning method.

Supervised learning may refer to a method for training an artificial neural network with training data that has been given a label. In addition, the label may refer to a target answer (or a result value) to be guessed by the artificial neural network when the training data is inputted to the artificial neural network. Unsupervised learning may refer to a method for training an artificial neural network using training data that has not been given a label. Reinforcement learning may refer to a learning method for training an agent defined within an environment to select an action or an action order for maximizing cumulative rewards in each state.

Machine learning of an artificial neural network implemented as a deep neural network (DNN) including a plurality of hidden layers may be referred to as deep learning, and the deep learning is one machine learning technique. Hereinafter, the meaning of machine learning includes deep learning.

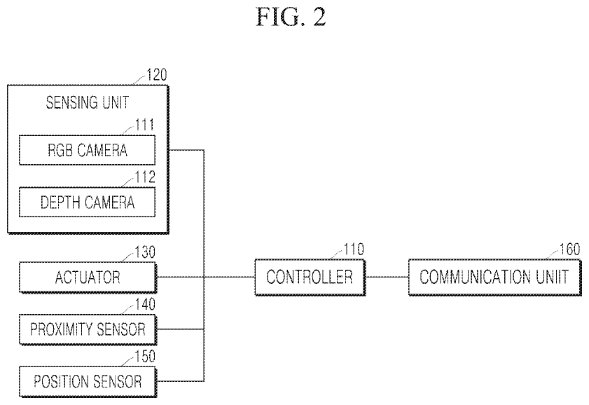

FIG. 2 is a view illustrating a structure of the wearable robot 100 according to an embodiment of the present disclosure. The robot 100 may include a controller 110, a sensing unit 120, and an actuator 130.

The controller 110 operates the actuator 130, and may control all operations of the robot 100 in the user wearing the robot 100, the user using the robot 100 (for example, moving while wearing the robot 100), or the user removing the robot 100.

The sensing unit 120 senses a position and pose of the user, and may transmit the sensed position and pose to the controller 110. The sensing unit 120 may include, for example, an RGB camera 111 and a depth camera 112.

The RGB camera 111 may acquire the position of the user by photographing an image. The depth camera 112 may acquire a distance and direction from the robot 100 to the joints of the user by photographing the image. By including the RGB camera 111 and the depth camera 112, the sensing unit 120 may acquire the position and pose of the user from a photographed image. The sensing unit 120 may include, for example, the RGB camera 111 and the depth camera 112, and may be provided as a Kinect sensor, which is a well-known technology.

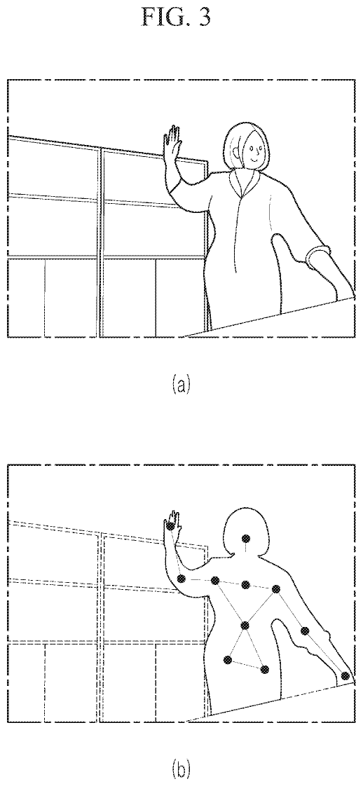

FIG. 3 is a view illustrating a function of the sensing unit 120 according to an embodiment of the present disclosure. The sensing unit 120 may simultaneously obtain a color image and a depth image in which the user is shown, by photographing the user by using the RGB camera 111 and the depth camera 112. FIG. 3A illustrates the color image of the user and FIG. 3B illustrates the depth image of the user.

The depth image may represent the frame of the user. Referring to FIG. 3B, the depth image obtained by photographing the user may represent the frame of the user by using dots and lines.

The controller 110 may acquire a distance and direction to the respective points in the depth image received from the sensing unit 120. Accordingly, the controller 110 may correctly acquire the pose of the user from the depth image. The depth image may be immediately obtained by the sensing unit 120 photographing the user without the help of an additional device.

The actuator 130 may operate in accordance with the command received from the controller 110, and may move positions of the joints of the robot 100. The actuator 130 may rotate the joints of the robot 100, and may change a pose of the robot 100.

Accordingly, by operating the actuator 130, the controller 110 may change the pose of the robot 100 to be suitable for a situation when the user wears the robot 100, uses the robot 100, or removes the robot 100.

The robot 100 may further include a plurality of proximity sensors 140. The proximity sensors 140 may be arranged in the robot 100 and connected to the controller 100, and may sense whether the respective parts of the robot 100 are within a set distance from the corresponding parts of the body of the user.

The proximity sensors 140 are provided in the respective parts of the robot 100, and may sense the body of the user when the body of the user is within a predetermined range. The proximity sensors 140 may sense the body of the user when the body of the user is within a certain distance therefrom.

The proximity sensor 140 may be, for example, an ultrasonic sensor using ultrasonic waves as a sensing means, an optical sensor using light, a capacitive sensor for measuring and sensing a dielectric constant of an object to be sensed, or a sensor using an electric field or a magnetic field.

When the body of the user approaches the robot 100, the proximity sensors 140 may sense the approach of the body of the user to the robot 100, and may transmit a sensing signal to the controller 110. For example, when the user wears the robot 100, the actuator 130 may operate and moves the robot 100, and accordingly the body parts of the user may approach the corresponding parts of the robot 100. When a degree of the approach is within a set range, the proximity sensors 140 may sense the approach and transmit the sensing signal to the controller 110, and the controller 110 may stop an operation of the actuator 130.

The robot 100 may further include position sensors 150 for sensing the position of the robot 100. The position sensor 150 may be, for example, a global positioning sensor (GPS). The robot 100 may acquire the position thereof through the position sensors 150.

For example, the position sensors 150 may be connected to the controller 110, and the controller 110 may receive information on the position of the robot 100 from the position sensors 150, and may represent the information as first coordinates. Here, the first coordinates may be world coordinates.

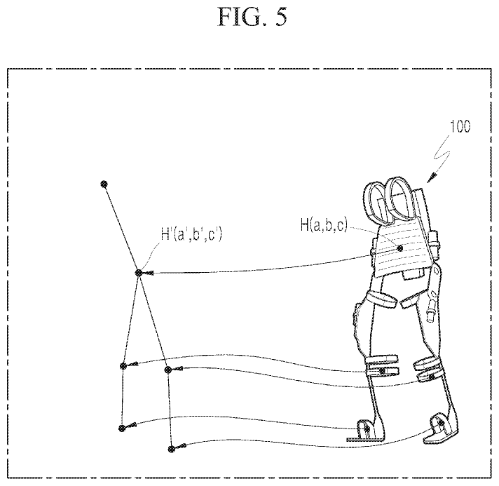

The position sensors 150 may be provided in the joints of the robot 100. Accordingly, the controller 110 may acquire the positions of the joints of the robot 100 as world coordinates, and may generate motion trajectories of the joints of the robot 100 based on the world coordinates. The motion trajectories will be described in detail with reference to FIG. 5.

The robot 100 may further include a communication unit 160, and the controller 110 may be connected to the terminal 200 and the server 300 through the communication unit 160.

The communication unit 160 may include at least one of a mobile communication module or a wireless Internet module. The communication unit 160 may further include a near field communication (NFC) module.

The mobile communication module may transmit and receive a wireless signal to and from at least one of a base station, the external terminal 200, and the server 300 in a mobile communication network established according to technical standards or communication methods for mobile communications (for example, Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), and Long Term Evolution-Advanced (LTE-A)), and 5G (fifth generation) communication.

The wireless Internet module, as a module for wireless Internet access, may be mounted in or outside the robot 100. The wireless Internet module may transmit and receive the wireless signal in a communication network in accordance with wireless Internet technologies.

The robot 100 may transmit and receive data to and from the server 300 and the terminal 200 capable of performing various communications through a 5G network. In particular, the robot 100 may perform data communications with the server 300 and the terminal 200 by using at least one network service among enhanced mobile broadband (eMBB), ultra-reliable and low latency communications (URLLC), and massive machine-type communications (mMTC).

The eMBB is a mobile broadband service, and provides, for example, multimedia contents and wireless data access. In addition, improved mobile services such as hotspots and broadband coverage for accommodating the rapidly growing mobile traffic may be provided via eMBB. Through a hotspot, the high-volume traffic may be accommodated in an area where user mobility is low and user density is high. Through broadband coverage, a wide-range and stable wireless environment and user mobility may be guaranteed.

A URLLC service defines requirements that are far more stringent than existing LTE in terms of reliability and delay of data transmission. Examples of URLLC services include 5G services for production process automation, telemedicine, remote surgery, transportation, and safety.

The mMTC is a transmission delay-insensitive service that requires a relatively small amount of data transmission. The mMTC enables a much larger number of terminals 300, such as sensors, than general mobile cellular phones to be simultaneously connected to a wireless access network. The communication module of the terminal 300 should be inexpensive, and there is a need for improved power efficiency and power saving technology capable of operating for years without battery replacement or recharging.

The user may command the robot 100 to be worn or removed, and the robot 100 may operate in accordance with the command of the user. At this time, the user may give a voice command to the robot 100 by using the terminal 200, or may directly give the voice command to the robot 100.

For example, the terminal 200 may transmit the command of the user on the wearing or removing of the robot 100 to the controller 110. An application for the user inputting the command on the robot 100 and transmitting the input command to the robot 100 may be provided in the terminal 200.

When the user inputs the command on the wearing or removing of the robot 100 to the terminal 200, and the terminal 200 transmits the command of the user to the controller 110, the controller 110 may control the actuator 130, which is required for the wearing or removing of the robot 100.

In another embodiment, the robot 100 may further include a microphone that is connected to the controller 110 and to which a voice of the user is inputted. The user may give the voice command on the wearing or removing of the robot 100 as a spoken utterance.

When the command of the user is inputted to the microphone, the controller 110 may receive, from the microphone, a signal on the command of the user. The controller 110 may receive the voice command of the user and control the actuator 130, which is required for the wearing or removing of the robot 100.

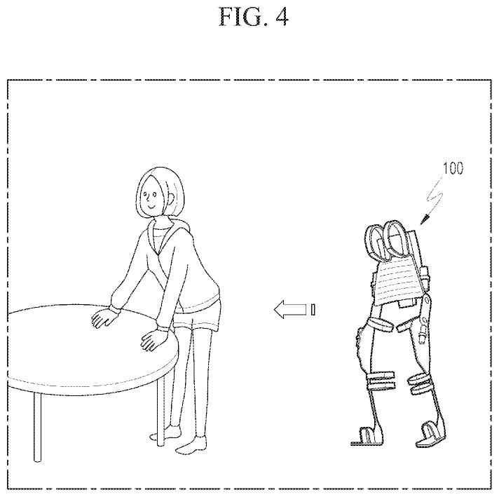

FIG. 4 is a view illustrating an operation of the wearable robot 100 according to an embodiment of the present disclosure. Hereinafter, a process of the user wearing the robot 100 will be described with reference to FIGS. 4 and 5. The user may give the voice command on the wearing of the robot 100 to the robot 100, and adopt a pose for wearing the robot 100 by leaning on a safe supporting means such as a table.

From a position away from the user by a certain distance, the robot 100 may move to approach the user when the robot 100 receives the command of the user on the wearing of the robot 100. At this time, for example, the robot 100 may move to the user by walking by itself.

In another embodiment, the robot 100 may be mounted on a moving device capable of moving to the user. Here, the robot 100 may be mounted on the moving device, and may be easily attached to and detached from the robot 100.

The moving device may communicate with the controller 110 of the robot 100, and a moving speed, a moving direction, and a moving position of the moving device may be controlled by the controller 110. While combined with the robot 100, the moving device may move toward the user, and when the user is wearing the robot 100, the moving device may be detached from the robot 100 and return to an original position.

Since a case in which the robot 100 moves to the user by being mounted on the moving device and is similar to a case in which the robot 100 moves to the user by walking, apart from the additional moving means, the detailed description below will be on the case in which the robot 100 moves to the user by walking. The case in which the robot 100 moves to the user by being mounted on the moving device will also be clearly derivable from the following description.

When the user gives a wearing command to the robot 100 and adopts a pose by leaning on the table, the sensing unit 120 provided in the robot 100 may photograph the user. Here, the RGB camera 111 provided in the sensing unit 120 may photograph the face of the user.

When it is difficult for the RGB camera 111 to photograph the face of the user due to a direction of the face of the user being different from that of the RGB camera 111, the user may change the direction of his or her face for a short period, and the RGB camera 111 may photograph the face of the user at this time.

The controller 110 may acquire personal information of the user from image information received from the RGB camera 111. That is, the controller 110 may acquire the personal information of the user from the facial image of the user.

The controller 110 may receive the personal information corresponding to the facial image of the user from the server 300 or the terminal 200. Even when a plurality of people are around the user, the robot 100 may check the personal information of the user through the facial image, and may correctly recognize the position of the user who calls the robot 100 among the plurality of people.

In addition, the controller 110 may obtain the depth image illustrated in FIG. 3B from an image of the user photographed by the depth camera 112 of the sensing unit 120, and may acquire the pose of the user, that is, the positions of the joints, from the depth image.

Here, the positions of the joints may be, for example, spatial distances and directions from the joints of the robot 100 to the corresponding joints of the user. Therefore, when the positions of the joints of the robot 100 from the depth image are represented as three-dimensional spatial coordinates, the spatial coordinates of the positions of the corresponding joints of the user may be acquired.

The positions of the joints of the user may be arbitrarily designated as positions to be acquired by the robot 100 in order for the user to wear the robot 100. For example, wrist joints, ankle joints, knee joints, the center of the pelvis, the center of the chest, and the center of the belly may be designated.

FIG. 5 is a view illustrating a method of generating motion trajectories of the joints of the wearable robot 100 according to an embodiment of the present disclosure.

The controller 110 may generate the motion trajectories of the joints of the robot 100 based on information on the position and pose of the user received from the sensing unit 120, in order to move the robot 100 such that the joints of the robot 100 are arranged at the positions of the corresponding joints of the user.

The robot 100 may acquire the positions of the joints of the robot 100 by using the provided position sensors 150. For example, referring to FIG. 5, among the joints of the robot 100, the position of the joint corresponding to the center of the belly of the user may be represented by three-dimensional spatial coordinates H(a, b, c). Here, H(a, b, c) is the world coordinates, for example, orthogonal coordinates.

The controller 110 may acquire the distance and direction to the position of the user by using the sensing unit 120, and may calculate a point H′ of the user, which corresponds to a point H of the robot 100, from the acquired distance and direction.

That is, the controller 110 may calculate second coordinates, for example, H′(a′, b′, c′) representing the position of the user, by compensating the distance and direction to the position of the user in the first coordinates, for example, H(a, b, c), representing the position of the robot 100 by using the position sensor 150.

By the above-described method, the robot 100 may acquire the second coordinates required for the user wearing the robot 100. The controller 110 may generate motion trajectories between the first coordinates and the second coordinates. The motion trajectories may be generated in accordance with the joints of the robot 100, and the number of motion trajectories may be properly selected so as not to hinder the user from wearing the robot 100.

The motion trajectories may be generated such that an obstacle that hinders the robot 100 from moving is avoided. In addition, the motion trajectories may be generated considering the walking characteristics of the robot 100. For example, since the robot 100 walks on two feet, it is appropriate to generate the motion trajectories with the structure of the robot 100 reflected so that the robot 100 stably walks without falling.

In addition, it is appropriate to generate the motion trajectories such that the first coordinates of the robot 100 coincide with the second coordinates of the user after the movement of the robot 100.

After the motion trajectories are generated, the controller 110 may operate the actuator 130, and the robot 100 may walk along the set motion trajectories toward the user. When the robot 100 moves toward the user by using the moving device, the controller 110 may give a moving command to the moving device, and may control the moving speed, the moving direction, and the moving position of the moving device. When the robot 100 moves toward the user, the actuator 130 may continuously move and may create a pose of the robot 100 corresponding to the pose of the user.

Using the proximity sensors 140, the robot 100 may acquire whether the robot 100 has reached the user and whether the joints of the robot 100 are within a set range of the user. That is, the robot 100 may acquire whether distances between the positions of the joints of the user and the positions of the corresponding joints of the robot 100 are within the set range. Here, the set range may be appropriately selected such the user is not hindered in wearing the robot 100.

When the respective parts of the robot 100 are within the set range, the controller 110 may stop the operation of the actuator 130. That is, the controller 110 may stop the operation of the actuator 130 so that the joints of the robot 100 no longer move, and the robot 100 may be worn by the user.

Alternatively, the user may wear the robot 100 by fixing the robot 100 to his or her body by using a fixing device provided in the robot 100. In another embodiment, the fixing device provided in the robot 100 may be connected to the actuator 130, and the fixing device may be fixed to the user by the actuator 130, and accordingly the robot 100 may be fixed to the body of the user.

When the robot 100 moves toward the user, the motion trajectories generated at first may be modified. It may be necessary to modify the motion trajectories when the user changes a pose while the robot 100 moves or an obstacle that does not exist at first appears in the moving path of the robot 100.

When the robot 100 moves toward the user, the sensing unit 120 may sense the position and pose of the user. That is, the sensing unit 120 may continuously photograph the user, and when the position or pose of the user changes or an obstacle appears in the moving path of the robot 100, the sensing unit 120 may sense the change or the obstacle and may transmit the sensing signal to the controller 110.

The controller 110 may modify the generated motion trajectories based on the information on the position and pose of the user, which are sensed by the sensing unit 120 during the movement of the robot 100. The controller 110 may modify the generated motion trajectories based on information on the generation of the obstacle in the moving path of the robot 100, which is sensed by the sensing unit 120.

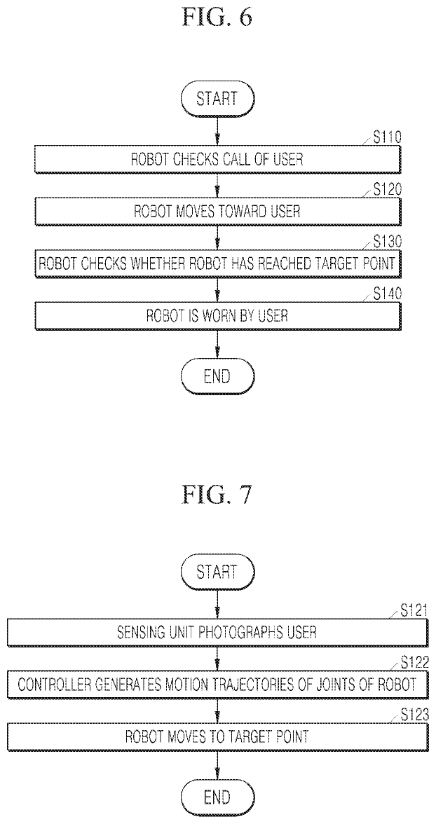

Hereinafter, a method of controlling the wearable robot 100 will be described with reference to FIGS. 6 to 11. In describing the method of controlling the wearable robot 100, description already given above may be omitted.

FIG. 6 is a view illustrating a method of controlling the wearable robot 100 according to an embodiment of the present disclosure.

In order to control the robot 100, in operation S110, the robot 100 may check a call of the user. The user may transmit a command on the wearing of the robot 100 via the terminal 200 or via voice, and the robot 100 may check the call of the user and proceed with the user wearing of the robot 100.

After the call is received from the user, the robot 100 may move toward the user in operation S120. The robot 100 may form the motion trajectories, and may move toward the user in accordance with the formed motion trajectories.

In operation S130, it may be determined whether the robot 100 has reached a target point. Here, the target point may be a point that the joints of the robot 100 reach immediately before the robot 100 is worn by the user, and may be properly set in order for the user to conveniently wear the robot 100. For example, the controller 110 may determine whether the joints of the robot 100 have reached the set target point from the position sensors 150 mounted in the joints of the robot 100.

When the robot 100 reaches the target point, in operation S140, the robot 100 may be worn by the user. In operation S140, the robot 100 may finely adjust the positions of the joints thereof by using the actuator 130. When the joints of the robot 100 are within the set range for the corresponding joints of the user, the robot 100 may be fixed to the user by using the fixing device.

Hereinafter, operations S120 and S140 will be described in more detail.

FIG. 7 is a view illustrating the operation S120 in which the robot 100 moves toward the user according to an embodiment of the present disclosure. In operation S120, the sensing unit 120 may photograph the user (S121).

Based on the image photographed by the sensing unit 120, the controller 110 may generate the motion trajectories of the joints of the robot 100 (S122). As described above, the sensing unit 120 may acquire the positions and directions of the joints of the user from the photographed image. The controller 110 may calculate the second coordinates by compensating the positions and directions of the joints of the user in the first coordinates representing the position of the robot 100. The controller 110 may generate motion trajectories using the first coordinates as a starting point and using the second coordinates as an ending point.

The robot 100 may move to the target point along the generated motion trajectories of the joints of the robot 100 (S123). As described above, when the robot 100 moves to the target point, in a case in which the user moves and the position or pose of the user changes, and in a case in which a new obstacle is found during the movement of the robot 100, the robot 100 may change the motion trajectories. The robot 100 may move along the changed motion trajectories.

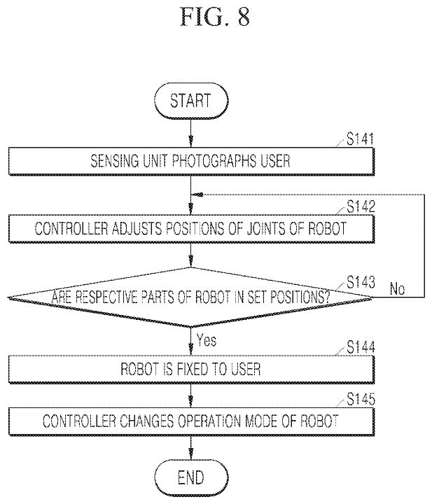

FIG. 8 is a view illustrating operation S140, in which the robot 100 is worn by the user according to an embodiment of the present disclosure. Operation S140 may be performed after operation S130 in which determination of whether the robot 100 has reached the target point is completed.

In operation S141, the sensing unit 120 may photograph the user at the target point. In the image photographed by the sensing unit 120, the controller 110 may acquire the distances between the positions of the joints of the robot 100 and the positions of the corresponding joints of the user.

Based on the photographed image, in operation S142, the controller 110 may adjust the positions of the joints of the robot 100 such that the joints of the robot 100 are arranged at the positions of the corresponding joints of the user. By finely adjusting the positions of the joints of the robot 100 by operating the actuator 130, the controller 110 may bring the joints of the robot 100 into closer proximity to the corresponding joints of the user.

The controller 110 may determine whether the respective parts of the robot 100 are within a set distance from the corresponding parts of the user. In operation S143, as described above, the controller 110 may determine whether the respective parts of the robot 100 are within the set distance from the corresponding parts of the user by using the proximity sensors 140.

When it is determined that the respective parts of the robot 100 are not within the set distance from the corresponding parts of the user, operation S142 may be performed continuously. When it is determined that the respective parts of the robot 100 are within the set distance from the corresponding parts of the user, the controller 110 may stop the operation of the actuator 130, and proceed with the next operation.

After operation S143 is completed, the robot 100 may be fixed to the user in operation S144. As described above, the user may directly fix himself or herself to the fixing device, or the fixing device may be fixed to the user by operating the actuator 130.

After the robot 100 is fixed to the user, in operation S145, the controller 110 may change an operation mode of the robot 100 so that the robot 100 follows the movement of the user.

The robot 100 may help the user walk. Accordingly, after the robot 100 is fixed to the user, when the user walks using the robot 100 or performs other movements, the robot 100 may move to follow the movement of the user. Therefore, the controller 110 may change the operation mode such that the robot 100 follows the movement of the user in a state in which the robot 100 itself moves.

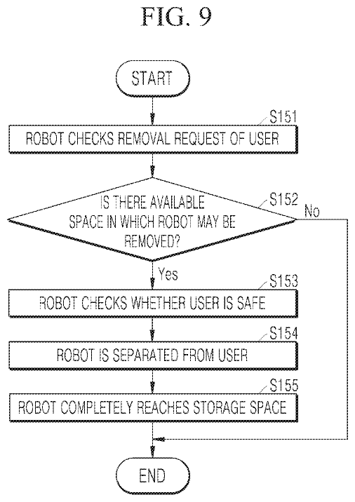

Hereinafter, a method of removing the robot 100 from the user in a state in which the robot 100 is being worn by the user will be described in detail. FIG. 9 is a view illustrating a method of the robot 100 being removed from the user according to an embodiment of the present disclosure.

The robot 100 may be removed from the user by a request of the user. The user may request the robot 100 to be removed while safely leaning on the supporting means such as the table or a wall, before removing the robot 100. At this time, the user may give a command on the removing of the robot 100 to the robot 100 via the terminal 200 or via voice.

In operation S151, the removal request of the user may be checked by the robot 100.

When the removal request is performed, in operation S152, the robot 100 may check whether there is an available space in which the robot 100 may be removed. For the convenience and safety of the user, a certain space is required when the robot 100 is removed. In addition, a space in which the robot 100 stored after being removed from the user and is required.

Therefore, the robot 100 may check whether there is an available space in which the robot 100 may be removed and stored while securing safety of the user. The robot 100 may check whether such a space is available by photographing a peripheral image by using the sensing unit 120.

When it is determined that there is no such space is available, the robot may not be removed from the user. The robot 100 may only be removed from the user when such a space is available.

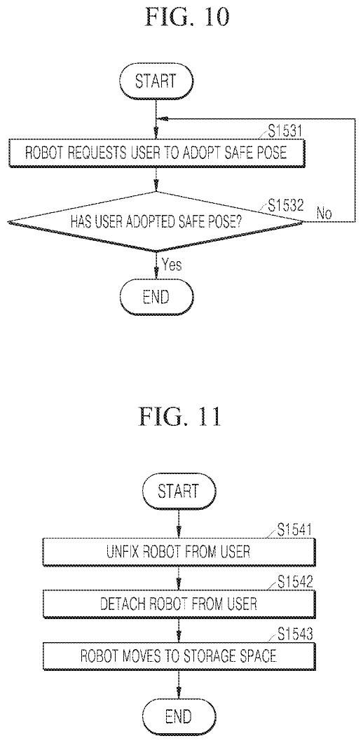

When there is a space in which the robot 100 may be removed and stored while securing the safety of the user, in operation S153, the robot 100 may check whether the user is safe. FIG. 10 is a view illustrating operation S153 of the robot 100 checking the safety of the user according to an embodiment of the present disclosure.

In operation S153, the robot 100 may request the user to adopt a safe pose (S1531). The safe pose may refer to a pose in which the safety of the user may be secured such that the user does not fall down after the robot 100 is removed, such as a state in which the user leans on the supporting means such as the table or is supported by another person.

The request of the robot 100 may be outputted via the terminal 200 or via voice through a speaker provided in the robot 100. The user may adopt the safe pose in accordance with the request of the robot 100 or before the request of the robot 100.

It may be then be determined whether the user has adopted the safe pose by the robot 100 analyzing the pose of the user (S1532). Since the user is still wearing the robot 100, the pose of the user coincides with the pose of the robot 100. Therefore, the robot 100 may acquire and analyze the pose of the user by using the position sensors 150 provided in the joints of the robot 100.

In addition, the robot 100 may check whether the user is leaning on the supporting means or is being supported by another person by photographing the peripheral image using the sensing unit 120.

When it is determined as a result of analysis that the user has not adopted the safe pose, operation S1531 and operation S1532 may be repeated. As a result of the user repeatedly changing the pose, when it is determined by the robot 100 that the user has adopted the safe pose, operation S154 may proceed.

In operation S154, the robot 100 may be separated from the user. FIG. 11 is a view illustrating operation S154 in which the robot 100 is separated from the user according to an embodiment of the present disclosure

In operation S154, the robot 100 may be unfixed from the user (S1541). At this time, the user may directly unfix the fixing device, or unfix the fixing device by operating the actuator 130.

After the robot 100 is unfixed from the user, the robot 100 may be detached from the user in operation S1542. The robot 100 may be moved away from the user by operating the actuator 130. In the process of detaching the robot 100 from the user, for the safety of the user, the robot 100 may acquire the distance between the respective parts of the body of the user and the respective corresponding parts of the robot 100 by using the proximity sensors 140.

The controller 110 may control the actuator 130 such that the robot 100 moves in a position and direction in which the user is safe, based on the distance between the respective parts of the body of the user and the respective parts of the robot 100, which are acquired by the proximity sensors 140.

After the robot 100 is detached from the user, the robot 100 may move to a storage space in operation S1543. Here, the storage space in which the detached robot 100 is stored may be, for example, a space in which a charging device capable of charging the robot 100 is arranged, or a space arbitrarily designated by the user. The user may designate the storage space for the robot 100 via the terminal 200 or via voice.

The robot 100 may walk by itself to the storage space, or may move to the storage space while being mounted on the moving device, when the moving device is provided. If required, the robot 100 may generate motion trajectories from a current position to the storage space after being detached from the user, and may move along the motion trajectories. Since the robot 100 generating the motion trajectories and moving along the motion trajectories has been described above, a repeat description thereof is omitted.

Referring back to FIG. 9, after the robot 100 is separated from the user, in operation S1551 the robot 100 may completely reach the storage space. When the charging device is provided in the storage space, the robot 100 may be charged with the required power while being stored. The stored robot 100 may perform the wearing process in accordance with the wearing command of the user.

According to embodiments of the present disclosure, since the wearable robot 100 may approach the user and may be worn by the user in accordance with the command of the user, the convenience of the user may improve.

According to the embodiments of the present disclosure, the user may wear the wearable robot 100 without being supported by another person.

According to the embodiments of the present disclosure, the user may remove the wearable robot 100 without being supported by another person.

According to the embodiments of the present disclosure, the wearable robot 100 may be safely worn by the user or removed from the user by using the sensing unit 120.

An AI device, an AI server, and an AI system according to an embodiment of the present disclosure will be described below.

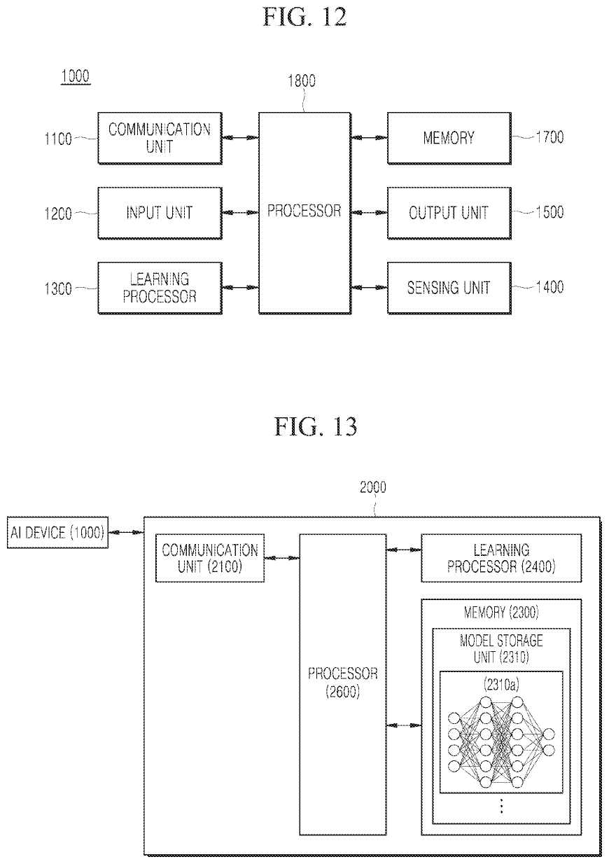

FIG. 12 is a diagram illustrating an artificial intelligence (AI) device 1000 according to an embodiment of the present disclosure.

The AI device 1000 may be implemented as a mobile or immobile device such as a TV, a projector, a mobile phone, a smartphone, a desktop computer, a notebook computer, a digital broadcast terminal, a personal digital assistant (PDA), a portable multimedia player (PMP), a navigation device, a tablet PC, a wearable device, a set-top box (STB), a digital media broadcasting (DMB) receiver, a radio, a washing machine, a refrigerator, a desktop computer, digital signage, a robot, and a vehicle.

Referring to FIG. 12, the AI device 1000 may include a communication unit 1100, an input unit 1200, a learning processor 1300, a sensing unit 1400, an output unit 1500, a memory 1700, and a processor 1800.

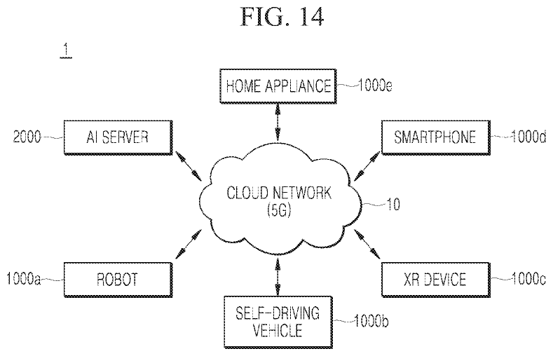

The communication unit 1100 may transmit or receive data with external devices, such as other AI devices (1000a to 1000e in FIG. 14) or an AI server 2000, using wired/wireless communications technology. For example, the communication unit 1100 may transmit or receive sensor data, a user input, a trained model, a control signal, and the like with the external devices.

In this case, the communications technology used by the communication unit 1100 may be technology such as global system for mobile communication (GSM), code division multi access (CDMA), long term evolution (LTE), 5G, wireless LAN (WLAN), Wireless-Fidelity (Wi-Fi), Bluetooth™, radio frequency identification (RFID), infrared data association (IrDA), ZigBee, and near field communication (NFC).

The input unit 1200 may obtain various types of data.

The input unit 1200 may include a camera for inputting an image signal, a microphone for receiving an audio signal, and a user input unit for receiving information inputted by a user. Here, the camera or the microphone may be treated as a sensor, and the signal obtained from the camera or the microphone may be referred to as sensing data or sensor information.

The input unit 1200 may obtain, for example, learning data for model learning and input data used when output is obtained using a learning model. The input unit 1200 may obtain raw input data. In this case, the processor 1800 or the learning processor 1300 may extract an input feature by preprocessing the input data.

The learning processor 1300 may train a model, composed of an artificial neural network, using learning data. Here, the trained artificial neural network may be referred to as a trained model. The trained model may be used to infer a result value with respect to new input data rather than learning data, and the inferred value may be used as a basis for a determination to perform an operation.

The learning processor 1300 may perform AI processing together with a learning processor 2400 of the AI server 2000.

The learning processor 1300 may include a memory which is combined or implemented in the AI device 1000. Alternatively, the learning processor 1300 may be implemented using the memory 1700, an external memory directly coupled to the AI device 1000, or a memory maintained in an external device.

The sensing unit 1400 may obtain at least one of internal information of the AI device 1000, surrounding environment information of the AI device 1000, or user information, by using various sensors.

The sensing unit 1400 may include sensors such as a proximity sensor, an illumination sensor, an acceleration sensor, a magnetic sensor, a gyroscope sensor, an inertial sensor, an RGB sensor, an infrared (IR) sensor, a fingerprint recognition sensor, an ultrasonic sensor, an optical sensor, a microphone, a light detection and ranging (lidar) sensor, and a radar.

The output unit 1500 may generate a visual, auditory, or tactile related output.

The output unit 1500 may include a display unit outputting visual information, a speaker outputting auditory information, and a haptic module outputting tactile information.

The memory 1700 may store data to support various functions of the AI device 1000. For example, the memory 1700 may store input data, training data, a trained model, and training history, acquired by the input unit 1200.

The processor 1800 may determine at least one executable operation of the AI device 1000 based on information determined or generated by using a data analysis algorithm or a machine learning algorithm. In addition, the processor 1800 may control components of the AI device 1000 to thereby perform the determined operation.

In order to do so, the processor 1800 may request, retrieve, receive, or use information or data of the learning processor 1300 or the memory 1700, and may control components of the AI device 1000 to thereby execute a predicted operation, or an operation determined to be preferable, among the determined at least one executable operation.

In this case, when connection with an external device is necessary in order to perform the determined operation, the processor 1800 may generate a control signal for controlling the corresponding external device, and may transmit the generated control signal to the corresponding external device.

The processor 1800 may obtain intent information about a user input, and determine a requirement of a user based on the obtained intent information.

The processor 1800 may obtain intent information corresponding to the user input by using at least one of a speech-to-text (STT) engine for converting a speech input into a character string or a natural language processing (NLP) engine for obtaining intent information of natural language.

The at least one of the STT engine or the NLP engine may be composed of artificial neural networks, at least some of which are trained according to a machine learning algorithm. In addition, the at least one of the STT engine or the NLP engine may be trained by the learning processor 1300, trained by the learning processor 2400 of the AI server 2000, or trained by distributed processing thereof.

The processor 1800 may collect history information including, for example, operation contents and user feedback on an operation of the AI device 1000, and may store the history information in the memory 1700 or the learning processor 1300, or transmit the history information to an external device such as an AI server 2000. The collected history information may be used to update a learning model.

The processor 1800 may control at least some of components of the apparatus 1000, in order to drive an application stored in the memory 1700. Furthermore, the processor 1800 may combine and operate at least two or more constituting elements among the constituting elements included in the AI device 1000, in order to operate the application program.

FIG. 13 is a diagram illustrating an artificial intelligence (AI) server 2000 according to an embodiment of the present disclosure.

Referring to FIG. 13, the AI server 2000 may refer to a device for training an artificial neural network using a machine learning algorithm or using a trained artificial neural network. Here, the AI server 2000 may include a plurality of servers to perform distributed processing, and may be defined as a 5G network. In this case, the AI server 2000 may be included as a part of the AI device 1000, and may thus perform at least a part of the AI processing together with the AI device 1000.

The AI server 2000 may include a communication unit 2100, a memory 2300, a learning processor 2400, and a processor 2600.

The communication unit 2100 may transmit and receive data with an external device such as the AI device 1000.

The memory 2300 may include a model storage unit 2310. The model storage unit 2310 may store a model (or an artificial neural network 2310a) which has been learned or is being learned via the learning processor 2400.

The learning processor 2400 may train the artificial neural network 2310a by using learning data. The learning model may be used while mounted in the AI server 2000 of the artificial neural network, or may be used while mounted in an external device such as the AI device 1000.

The learning model may be implemented as hardware, software, or a combination of hardware and software. When a portion or the entirety of the learning model is implemented as software, one or more instructions, which constitute the learning model, may be stored in the memory 2300.

The processor 2600 may infer a result value with respect to new input data by using the learning model, and generate a response or control command based on the inferred result value.

FIG. 14 is a diagram illustrating an AI system 1 according to an embodiment of the present disclosure.

Referring to FIG. 14, in the AI system 1, at least one of an AI server 2000, a robot 1000a, a self-driving vehicle 1000b, an XR device 1000c, a smartphone 1000d, or a home appliance 1000e are connected to a cloud network 10. Here, the robot 1000a, the self-driving vehicle 1000b, the XR device 1000c, the smartphone 1000d, or a home appliance 1000e, to which AI technology has been applied, may be referred to as an AI device 1000a to 1000e.

A cloud network 10 may comprise part of a cloud computing infrastructure, or refer to a network existing in cloud computing infrastructure. Here, the cloud network 10 may be constructed by using a 3G network, a 4G or Long Term Evolution (LTE) network, or a 5G network.

In other words, the devices 1000a to 1000e and 2000 constituting the AI system 1 may be connected to each other through the cloud network 10. In particular, each individual device 1000a to 1000e and 2000 may communicate with each other through a base station, but may also communicate directly to each other without relying on the base station.

The AI server 2000 may include a server performing AI processing and a server performing computations on big data.

The AI server 2000 may be connected to at least one of the robot 1000a, the self-driving vehicle 1000b, the XR device 1000c, the smartphone 1000d, or the home appliance 1000e, which are AI devices constituting the AI system 1, through the cloud network 10, and may assist with at least a part of the AI processing conducted in the connected AI devices 1000a to 1000e.

At this time, the AI server 2000 may train the artificial neural network according to a machine learning algorithm instead of the AI devices 1000a to 1000e, and may store the learning model or transmit the learning model to the AI devices 1000a to 1000e.

At this time, the AI server 2000 may receive input data from the AI devices 1000a to 1000e, infer a result value from the received input data by using the learning model, generate a response or control command based on the inferred result value, and transmit the generated response or control command to the AI devices 1000a to 1000e.

In addition, the AI devices 1000a to 1000e may infer a result value from the input data directly by employing the learning model, and generate a response or control command based on the inferred result value.

While the invention has been explained in relation to its embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A robot, comprising:
a controller;
a sensing unit configured to sense a position and pose of a user and transmit the sensed position and pose to the controller; and
an actuator configured to receive a command from the controller, operate, and move positions of joints of the robot,
wherein the controller is configured to generate motion trajectories of the joints of the robot in order to move the joints of the robot to be arranged at positions of corresponding joints of the user, based on information on the position and pose of the user received from the sensing unit.

2. The robot of claim 1, wherein the sensing unit comprises an RGB camera configured to acquire the position of the user, and a depth camera configured to acquire distances from the robot to the joints of the user.

3. The robot of claim 2,
wherein the RGB camera is configured to photograph the face of the user, and
wherein the controller is configured to acquire personal information of the user from image information received from the RGB camera.

4. The robot of claim 1,
wherein the sensing unit is configured to sense the position and pose of the user when the robot moves toward the user, and
wherein the controller is configured to modify the generated motion trajectories based on the information on the position and pose of the user sensed by the sensing unit, during the movement of the robot.

5. The robot of claim 1, further comprising proximity sensors arranged in the robot and connected to the controller so as to sense whether the respective parts of the robot are within a set distance from corresponding parts of the body of the user,
wherein the controller is configured to stop an operation of the actuator when the respective parts of the robot are within the set distance from the corresponding parts of the body of the user.

6. The robot of claim 1, wherein the robot moves to the position of the user by walking by itself.

7. The robot of claim 1, wherein the robot moves to the position of the user by being mounted on a moving device.

8. The robot of claim 1,
wherein the robot further comprises a position sensor configured to sense the position of the robot, and
wherein the controller compensates a distance and direction to the position of the user in first coordinates representing the position of the robot, calculates second coordinates representing the position of the user, and generates motion trajectories between the first coordinates and the second coordinates.

9. The robot of claim 1, further comprising a communication unit,
wherein the controller is connected to a terminal through the communication unit, and
wherein the terminal transmits, to the controller, a command of the user on the wearing or removing of the robot.

10. The robot of claim 1, further comprising a microphone that is connected to the controller and to which a voice of the user is inputted,
wherein the controller receives a voice command of the user and proceeds with the wearing or removing of the robot by the user.

11. A method of controlling a robot, the method comprising:
the robot checking a call of a user;
the robot moving toward the user;
checking whether the robot has reached a target point; and
the user wearing the robot,
wherein the robot comprises:
a controller; and
a sensing unit configured to sense a position and pose of a user and transmit the sensed position and pose to the controller, and
wherein the controller is configured to generate motion trajectories of the joints of the robot in order to move the joints of the robot to be arranged at positions of corresponding joints of the user, based on information on the position and pose of the user received from the sensing unit.

12. The method of claim 11, wherein the robot moving toward the user comprises:
the sensing unit photographing the user;
the controller generating motion trajectories of joints of the robot based on an image photographed by the sensing unit; and
the robot moving to a target point along the generated motion trajectories of the joints of the robot.

13. The method of claim 11, wherein the user wearing the robot comprises:
the sensing unit photographing the user at the target point;
the controller adjusting the positions of the respective joints of the robot such that the joints of the robot are arranged at the positions of the corresponding joints of the user, based on the photographed image;
the controller checking whether the respective parts of the robot are within a set distance from the corresponding parts of the body of the user;
the robot being fixed to the user; and
the controller changing an operation mode of the robot so that the robot follows an operation of the user.

14. The method of claim 11, wherein the robot is removed from the user by a request of the user, and wherein the removing of the robot comprises:
the robot checking the removal request of the user;
the robot checking whether there is an available space for the robot to be removed;
the robot checking whether the user is safe;
the robot being separated from the user; and
the robot completely reaching a storage space.

15. The method of claim 14, wherein the robot checking whether the user is safe comprises:
the robot requesting the user to adopt a safe pose; and
the robot analyzing the pose of the user and checking whether the user has adopted the safe pose.

16. The method of claim 14, wherein the robot being separated from the user comprises:
unfixing the robot from the user;
detaching the robot from the user; and
the robot moving to the storage space.
